(19) United States
(12) Patent Application Publication
(10) Pub. No.: US 2025/0322565 A1
(43) Pub. Date: Oct. 16, 2025

(54) PET PARAMETER DETERMINATION METHOD AND APPARATUS, AND DEVICE AND STORAGE MEDIUM

(71) Applicant: SHENZHEN INSTITUTES OF ADVANCEDTECHNOLOGY CHINESE ACADEMY OF SCIENCES, Shenzhen (CN)

(72) Inventors: Tao SUN, Shenzhen (CN); Zhenguo WANG, Shenzhen (CN)

(73) Assignee: SHENZHEN INSTITUTES OF ADVANCEDTECHNOLOGY CHINESE ACADEMY OF SCIENCES, Shenzhen (CN)

(21) Appl. No.: 19/218,995

(22) Filed: May 27, 2025

Related U.S. Application Data
(63) Continuation of application No. PCT/CN2023/133057, filed on Nov. 21, 2023.

(30) Foreign Application Priority Data
Nov. 24, 2022 (CN) 202211485920.8

(51) Int. Cl. G06T 11/00; A61B 6/03; A61B 6/50; G06T 7/20

(52) U.S. Cl. CPC G06T 11/005; A61B 6/037; A61B 6/507; G06T 7/20; G06T 2207/10104; G06T 2207/30104; G06T 2210/41

(57) ABSTRACT

Disclosed are a PET parameter determination method and apparatus, and a device and a storage medium. Comprises: extracting a tracer identifier from the PET scanning data; performing image reconstruction on the PET scanning data, so as to obtain a PET image set; according to the PET image set, determining a sampling time activity curve corresponding to each pixel, and according to the tracer identifier and a pre-created correlation between a tracer identifier and a tissue compartmental model, determining a tissue compartmental model corresponding to the sampling time activity curve; on the basis of the tissue compartmental model, modifying an activity addition expression corresponding to the intensity of each pixel point corresponding to the tissue compartmental model, so as to update the activity addition expression; and according to the updated activity addition expression, determining the numerical value of at least one dynamic parameter corresponding to the PET image set.

### CROSS-REFERENCE TO RELATED APPLICATIONS

The application claims priority to Chinese patent application No. 202211485920.8, filed on Nov. 24, 2022, the entire contents of which are incorporated herein by reference.

### TECHNICAL FIELD

Embodiments of the present disclosure relate to the field of image processing, and in particular to a PET parameter determination method and apparatus, and a device and a storage medium.

### BACKGROUND

The dynamic images collected by whole-body PET (Positron Emission Computed Tomography) are of high quality, facilitating more precise parameter estimation. However, due to the greater number of pixel points in the whole-body PET compared to the traditional PET, the computational cost of using the conventional nonlinear estimation methods for determining the PET parameters is relatively high. Additionally, PET parameter determination based on linear regression of graphical estimation methods requires the tracer to reach a steady-state equilibrium in the blood and among tissues in vivo, which may lead to significant parameter estimation errors. Therefore, there is a need for an accurate method to calculate PET parameters that can enhance the speed of PET parameter determination.

### SUMMARY

The present disclosure provides a PET parameter determination method, apparatus, and a device and a storage medium to solve the problem of slower speed of the existing parameter determination method.

According to an aspect of the present disclosure, there is provided a PET parameter determination method, the method including:
acquiring PET scanning data of a scanned part, and extracting a tracer identifier from the PET scanning data;
performing image reconstruction on the PET scanning data, so as to obtain a PET image set;
according to the PET image set, determining a sampling time activity curve corresponding to each pixel, and according to the tracer identifier and a pre-created correlation between a tracer identifier and a tissue compartmental model, determining a tissue compartmental model corresponding to the sampling time activity curve;
on the basis of the tissue compartmental model, modifying an activity addition expression corresponding to the intensity of each pixel point corresponding to the tissue compartmental model, so as to update the activity addition expression; and
according to the updated activity addition expression, determining the numerical value of at least one dynamic parameter corresponding to the PET image set, wherein the dynamic parameter includes a flow velocity between tissue compartments in the tissue compartmental model and/or a net inflow rate of a tracer.

According to another aspect of the present disclosure, there is provided a PET parameter determination apparatus, the apparatus including:
an acquisition module, configured to acquire PET scanning data of a scanned part, and extract a tracer identifier from the PET scanning data;
an image reconstruction module, configured to perform image reconstruction on the PET scanning data, so as to obtain a PET image set;
a model determination module, configured to, according to the PET image set, determine a sampling time activity curve corresponding to each pixel, and according to the tracer identifier and a pre-created correlation between a tracer identifier and a tissue compartmental model, determine a tissue compartmental model corresponding to the sampling time activity curve;
a model updating module, configured to, on the basis of the tissue compartmental model, modify an activity addition expression corresponding to the intensity of each pixel point corresponding to the tissue compartmental model, so as to update the activity addition expression; and
a parameter determination module, configured to, according to the updated activity addition expression, determine the numerical value of at least one dynamic parameter corresponding to the PET image set, wherein the dynamic parameter includes a flow velocity between tissue compartments in the tissue compartmental model and/or a net inflow rate of a tracer.

According to another aspect of the present disclosure, there is provided an electronic device, including:
at least one processor; and
a memory communicatively connected with the at least one processor; wherein,
the memory stores a computer program executable by the at least one processor, the computer program being executed by the at least one processor to enable the at least one processor to perform the PET parameter determination method according to any one of the embodiments of the present disclosure.

According to another aspect of the present disclosure, there is provided a computer-readable storage medium having stored thereon computer instructions for causing a processor to implement the PET parameter determination method according to any one of the embodiments of the present disclosure.

The technical solution of an embodiment of the present disclosure is as follows: acquiring PET scanning data of a scanned part, and extracting a tracer identifier from the PET scanning data; performing image reconstruction on the PET scanning data, so as to obtain a PET image set; according to the PET image set, determining a sampling time activity curve corresponding to each pixel, and according to the tracer identifier and a pre-created correlation between a tracer identifier and a tissue compartmental model, determining a tissue compartmental model corresponding to the sampling time activity curve; on the basis of the tissue compartmental model, modifying an activity addition expression corresponding to the intensity of each pixel point corresponding to the tissue compartmental model, so as to update the activity addition expression; according to the updated activity addition expression, determining the numerical value of at least one dynamic parameter corresponding to the PET image set, wherein the dynamic parameter includes a flow velocity between tissue compartments in the tissue compartmental model and/or a net inflow rate of a tracer. On the basis of a tissue compartmental model and a corresponding activity addition expression, a PET parameter of a PET image is determined by using a linear estimation method, thereby improving the speed of estimation of the PET parameter.

It should be understood that what is described in this section is not intended to identify key or critical features of embodiments of the disclosure, nor is it intended to limit the scope of the disclosure. Other features of the present disclosure will become apparent from the following description.

### BRIEF DESCRIPTION OF DRAWINGS

To illustrate the technical solutions in the embodiments of the present disclosure more clearly, the accompanying drawings required for use in the description of the embodiments will be briefly described below, and it is obvious that the accompanying drawings in the following description are only some embodiments of the present disclosure, and other drawings can be obtained from these drawings for those skilled in the art without inventive step.

FIG. 1 is a flowchart of a PET parameter determination method according to an embodiment of the present disclosure;

FIG. 2 is a flowchart of another PET parameter determination method according to an embodiment of the present disclosure;

FIG. 3 is a flowchart of yet another PET parameter determination method according to an embodiment of the present disclosure;

FIG. 4 is a block diagram of a PET parameter determination apparatus according to an embodiment of the present disclosure; and

FIG. 5 is a block diagram of an electronic device according to an embodiment of the present disclosure.

### DETAILED DESCRIPTION OF THE EMBODIMENTS

In order for those skilled in the art to better understand the present solutions, the technical solutions in the embodiments of the present disclosure will be clearly and completely described below in conjunction with the accompanying drawings in the embodiments of the present disclosure, it is obvious that the described embodiments are only a part of embodiments of the present disclosure, rather than all of the embodiments. Based on the embodiments in the present disclosure, all other embodiments obtained by those of ordinary skill in the art without making inventive labor should belong to the scope of protection of the present disclosure.

It should be noted that the terms “first” and “second” and the like in the description and claims of the present disclosure and the figures above are used for distinguishing between similar objects and not necessarily for describing a particular sequential or chronological order. It should be understood that the data so used may be interchangeable where appropriate so that the embodiments of the disclosure described herein can be practiced in an order other than those illustrated or described herein. Furthermore, the terms “including” and “having” and any variations thereof are intended to cover a non-exclusive inclusion, for example, a process, method, system, product, or device including a series of steps or units is not necessarily limited to those steps or units clearly listed, but may include other steps or units not clearly listed or inherent to such processes, methods, products, or devices.

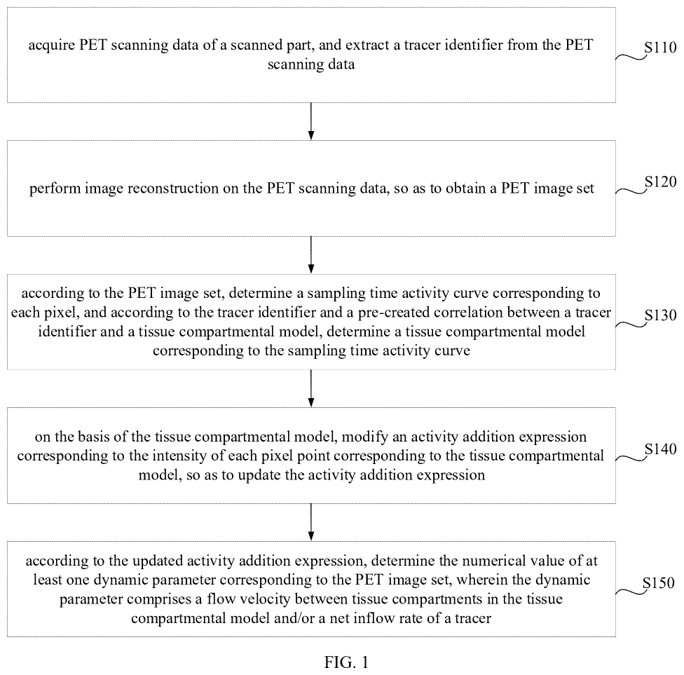

FIG. 1 is a flowchart of a PET parameter determination method according to an embodiment of the present disclosure, which is applicable to a PET parameter determination scenario. The method may be performed by a PET parameter determination apparatus, which may be implemented in hardware and/or software, or may be configured in an electronic device.

As shown in FIG. 1, the PET parameter determination method includes the following steps:
S110, PET scanning data of a scanned part is acquired, and a tracer identifier is extracted from the PET scanning data.

Wherein, the PET scanning data is scanning data of any part of the human body or the whole body.

PET is a novel imaging technology that can uniquely visualize the metabolism of biomolecules, receptor and neurotransmitter activities in vivo. It is utilized in the diagnosis and differential diagnosis of various diseases, assessment of disease severity, evaluation of treatment efficacy, research on organ functions, and development of new drugs. PET employs annihilation radiation and positron collimation (or photon collimation) techniques to measure the spatial distribution, quantity, and dynamic changes of a tracer or its metabolite molecules in vivo. This allows for the acquisition of imaging information regarding biochemical, physiological, and functional metabolic changes resulting from the interaction between PET tracers and targets (such as receptors, enzymes, ion channels, antigenic determinants, and nucleic acids) at the molecular level in vivo.

The tracer identifier refers to the name or code of the tracer. The tracer is a marker added to observe, study, and measure the behavior or properties of a substance in a specified process. In an embodiment, the tracer identifier may be an existing tracer identifier, such as a glucose metabolism tracer identifier (18F-FDG), a prostate cancer radioactive tracer identifier (68Ga-PSMA), or a 18F-FAPI (18F-fibroblast activation protein inhibitor).

In a particular embodiment, the PET scanning data is obtained by a PET-CT scanner, which may be, for example, a uEXPLORER PET-CT scanner. First, a CT scan is performed on a subject for attenuation correction. Following an intravenous injection of 18F-FDG into the vein in the lower extremity, a 60-minute PET list-mode acquisition is initiated. Subsequently, the 0-60 minute scanning data is divided into 66 PET scanning data subsets, including 5 seconds×24 frames, 10 seconds×6 frames, 30 seconds×6 frames, 60 seconds×6 frames, and 120 seconds×24 frames.

S120, image reconstruction is performed on the PET scanning data, so as to obtain a PET image set.

Image reconstruction is performed on the PET scanning data using an image reconstruction algorithm to obtain corresponding PET images, thereby obtaining a PET image set.

The image reconstruction algorithm includes an iterative image reconstruction algorithm, a GPU-accelerated particle filter PET image reconstruction algorithm, a PET image reconstruction algorithm based on a dilated U-Net neural network, a PET image reconstruction algorithm based on anisotropic diffusion filtering and nonlocal prior, a sinogram-based reconstruction algorithm, and the like. Wherein, the sinogram-based reconstruction algorithm includes a filtered back projection (FBP) method, a maximum likelihood expectation maximization (MLEM) method and an ordered subset expectation maximization (OSEM) method. Wherein, the FBP method is a reconstruction algorithm that performs filtering processing by a filter function before back-projection. The MLEM method is an iterative image reconstruction algorithm based on maximum likelihood estimation, which updates pixel estimates using the expectation maximization algorithm. Each update increases the likelihood function, finally the approximation of the likelihood function is converged to its maximum, thereby obtaining the maximum likelihood estimate for each pixel. Since all measured data are used to update pixel estimates each time, this method is relatively slow. The OSEM method is an iterative image reconstruction algorithm based on the maximum likelihood expectation method, which divides all projection data into a plurality of subsets. Each time a subset of data is used, all pixels are updated once. One complete cycle through all subsets constitutes one iteration. Specifically, first, an expression for calculating a conditional expectation value of a likelihood function is determined. Then, the pixel update value that maximizes the conditional expectation value of the likelihood function is derived using the method of finding extrema via derivatives. The value of the likelihood function after each pixel update is greater than or equal to the previous value, and the pixel values ultimately converge to maximize the likelihood function.

In a specific embodiment, image reconstruction is performed on the PET scanning data by using an existing image reconstruction algorithm, for example, a 3D ordered subset OSEM algorithm, which may be built into the uEXPLORER PET-CT scanner control system, reconstructs each PET scanning data subset into a 192×192×673 image matrix with a voxel size of 3.125×3.125×2.866 mm³. The image reconstruction involves 3 iterations, 28 subsets and 2 mm Gaussian smoothing. Additionally, attenuation and scatter corrections are applied based on the CT-based attenuation correction images.

Further, the image in the PET image set is an image with a standard uptake value.

The standard uptake value (SUV) refers to the ratio of radioactivity of the tracer taken up in a local tissue to the average injected activity in the whole body, for example, the ratio of radioactivity uptake at the lesion site to the average uptake in the whole body. In addition to factors such as the blood glucose level, the subject size, the lesion size, the delineation of the region of interest, the post-injection imaging time, and the clearance rate of ¹⁸F-FDG in the blood circulation, SUV is affected by factors such as the device performance, the imaging conditions, the acquisition mode, the reconstruction algorithm, and the attenuation correction.

S130, according to the PET image set, a sampling time activity curve corresponding to each pixel is determined, and according to the tracer identifier and a pre-created correlation between a tracer identifier and a tissue compartmental model, a tissue compartmental model corresponding to the sampling time activity curve is determined.

A time-activity curve (TAC) is a curve reflecting the concentration of a radioactive tracer within a region, with the vertical axis representing concentration and the horizontal axis representing time, and can be, for example, a curve reflecting the concentration of a radioactive tracer in tissue, plasma, or other regions of interest.

The tissue compartmental model may be selected from an irreversible two-tissue compartmental model, a reversible two-tissue compartmental model, a reversible one-tissue compartmental model, and the like. Wherein, the tracer identifier corresponding to the irreversible two-tissue compartmental model is optionally 18F-FDG (fluorodeoxyglucose, with the full chemical name being 2-fluoro-2-deoxy-D-glucose).

S140, on the basis of the tissue compartmental model, an activity addition expression corresponding to the intensity of each pixel point corresponding to the tissue compartmental model is modified, so as to update the activity addition expression.

Specifically, the activity addition of all compartments in the tissue compartmental model is represented by using the intensity of each pixel point of the PET image, and the activity addition expression is updated based on the tissue compartmental model to obtain an updated activity addition expression.

S150, according to the updated activity addition expression, the numerical value of at least one dynamic parameter corresponding to the PET image set is determined, wherein the dynamic parameter includes a flow velocity between tissue compartments in the tissue compartmental model and/or a net inflow rate of a tracer.

According to the updated activity addition expression, calculations are performed using a linear estimation method to obtain the flow velocity between the tissue compartments and/or the net inflow rate of the tracer.

Further, the method includes determining an image corresponding to the numerical value of the at least one dynamic parameter, respectively, to obtain at least one dynamic parameter image corresponding to the PET image set.

In particular, corresponding images are determined based on the dynamic values, i.e., a K₁ image, a k₂ image and a k₃ image corresponding to the flow velocities K₁, k₂ and k₃ between the tissue compartments and/or a Kᵢ image corresponding to the net glucose metabolic rate of the tissue organ.

The technical solution of an embodiment of the present disclosure is as follows: acquiring PET scanning data of a scanned part, and extracting a tracer identifier from the PET scanning data; performing image reconstruction on the PET scanning data, so as to obtain a PET image set; according to the PET image set, determining a sampling time activity curve corresponding to each pixel, and according to the tracer identifier and a pre-created correlation between a tracer identifier and a tissue compartmental model, determining a tissue compartmental model corresponding to the sampling time activity curve; on the basis of the tissue compartmental model, modifying an activity addition expression corresponding to the intensity of each pixel point corresponding to the tissue compartmental model, so as to update the activity addition expression; according to the updated activity addition expression, determining the numerical value of at least one dynamic parameter corresponding to the PET image set, wherein the dynamic parameter includes a flow velocity between tissue compartments in the tissue compartmental model and/or a net inflow rate of a tracer. On the basis of a tissue compartmental model and a corresponding activity addition expression, a PET parameter of a PET image is determined by using a linear estimation method, thereby improving the speed of estimation of the PET parameter.

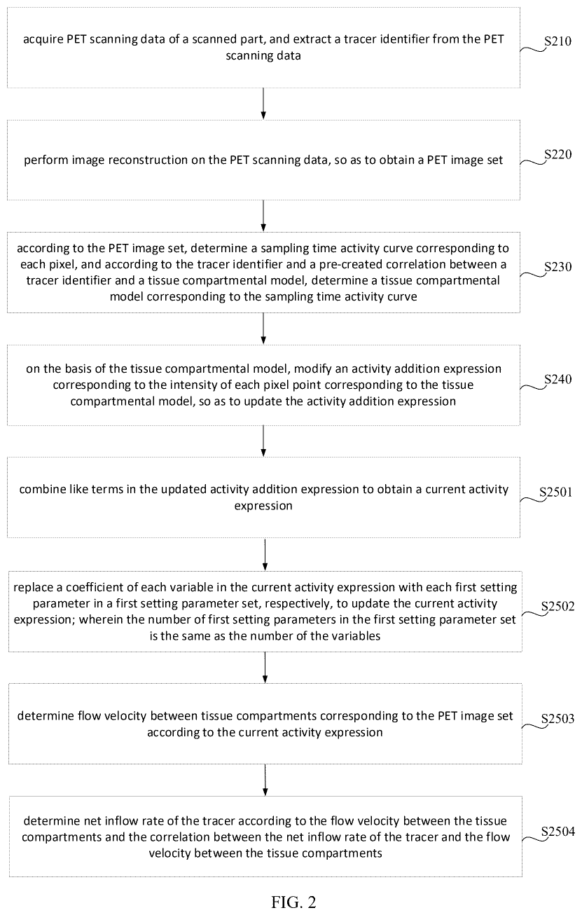

FIG. 2 is a flowchart of another PET parameter determination method according to an embodiment of the present disclosure, this embodiment belongs to the same inventive concept as the PET parameter determination method in the above-described embodiment, on the basis of which the process of determining the value of at least one dynamic parameter corresponding to the PET image set based on the updated activity addition expression is further described.

As shown in FIG. 2, the PET parameter determination method includes the following steps:

S210, PET scanning data of a scanned part is acquired, and a tracer identifier is extracted from the PET scanning data.

S220, image reconstruction is performed on the PET scanning data, so as to obtain a PET image set.

S230, according to the PET image set, a sampling time activity curve corresponding to each pixel is determined, and according to the tracer identifier and a pre-created correlation between a tracer identifier and a tissue compartmental model, a tissue compartmental model corresponding to the sampling time activity curve is determined.

In one embodiment, taking the tracer identifier as 18F-FDG and the tissue compartmental model as an irreversible two-tissue compartmental model as an example, a detailed explanation of the technical solution is provided, wherein the irreversible two-tissue compartmental model can be described by a set of linear ordinary differential equations:

$$\frac{{dC}_{1}}{dt} = {K}_{1} {C}_{p} ( t ) - ( {k}_{2} + {k}_{3} ) {C}_{1} ( t ) + {k}_{4} {C}_{2} ( t ) , \frac{{dC}_{2} ( t )}{dt} = {k}_{3} {C}_{1} ( t ) - {k}_{4} {C}_{2} ( t ) \qquad ( 1 )$$

wherein, Cp(t), C1(t) and C2(t) correspond to the activity of FDG in blood, the activity of free FDG and the activity of phosphorylated FDG, respectively. K₁, k₂, k₃ and k₄ represent flow rates between the tissue compartments. Since the phosphorylation process is an irreversible process in most tissues, the k₄ in the irreversible two-tissue compartmental model is 0.

S240, on the basis of the tissue compartmental model, an activity addition expression corresponding to the intensity of each pixel point corresponding to the tissue compartmental model is modified, so as to update the activity addition expression.

First, from the images of the PET image set, a 10 mm×10 mm×20 mm region is delineated in the added images of the early phase (0-30 s), and the blood input function Cₚ(t) is obtained at the ascending aortic arch. Based on the set of the linear ordinary differential equations (1) of the irreversible two-tissue compartmental model, the intensity of each pixel point of the PET image represents the addition of the activities of all the compartments, as detailed below:

$${C}_{T} ( t ) = CBV \cdot {C}_{p} ( t ) + {C}_{1} ( t ) + {C}_{2} ( t ) \qquad ( 2 )$$

wherein, C_{T}(t) is the measured activity in the tissue, Cₚ(t) is the activity in the blood, CBV is the blood volume fraction. Then, based on Equation (1), Equation (2) is rewritten to obtain the following equation:

$$\frac{{dC}_{T} ( t )}{dt} = CBV \frac{{dC}_{p} ( t )}{dt} + {K}_{1} {C}_{p} ( t ) - {k}_{2} {C}_{1} ( t ) \qquad ( 3 )$$

Equation (3) is rearranged to obtain an expression for C₁(t) as follows:

$${C}_{1} ( t ) = \frac{{K}_{1}}{{k}_{2}} \cdot {C}_{p} ( t ) + \frac{CBV}{{k}_{2}} \cdot \frac{{dC}_{p} ( t )}{dt} - \frac{1}{{k}_{2}} \cdot \frac{{dC}_{T} ( t )}{dt} \qquad ( 4 )$$

then, C₁(t) is substituted into Equation (2), and the result is as follows:

$${C}_{T} ( t ) = CBV \cdot {C}_{p} ( t ) + [ {C}_{2} ( t ) + \frac{{K}_{1}}{{k}_{2}} \cdot {C}_{p} ( t ) + \frac{CBV}{{k}_{2}} \cdot \frac{{dC}_{p} ( t )}{dt} - \frac{1}{{k}_{2}} \cdot \frac{{dC}_{T} ( t )}{dt} ] \qquad ( 5 )$$

an expression for C₂(t) is obtained according to Equation (5), as detailed below:

$${C}_{2} ( t ) = {C}_{T} ( t ) - CBV \cdot {C}_{p} ( t ) - \frac{{K}_{1}}{{k}_{2}} \cdot {C}_{p} ( t ) - \frac{CBV}{{k}_{2}} \cdot \frac{{dC}_{p} ( t )}{dt} + \frac{1}{{k}_{2}} \cdot \frac{{dC}_{T} ( t )}{dt} \qquad ( 6 )$$

based on Equation (2) in the set of the linear ordinary differential equations (1), and C₁(t) and C₂(t), the following equation is obtained:

$$\frac{{dC}_{T} ( t )}{dt} - CBV \cdot \frac{{dC}_{p} ( t )}{dt} - \frac{{K}_{1}}{{k}_{2}} \cdot \frac{{dC}_{p} ( t )}{dt} - \frac{1}{{k}_{2}} CBV \frac{{d}^{2} {C}_{p} ( t )}{{dt}^{2}} + \frac{1}{{k}_{2}} \frac{{d}^{2} {C}_{T} ( t )}{{dt}^{2}} = {k}_{3} [ \frac{{K}_{1}}{{k}_{2}} \cdot {C}_{p} ( t ) + \frac{CBV}{{k}_{2}} \frac{{dC}_{p} ( t )}{dt} - \frac{1}{{k}_{2}} \frac{{dC}_{T} ( t )}{dt} ] \qquad ( 7 )$$

S2501, like terms in the updated activity addition expression are combined to obtain a current activity expression.

Like terms in Equation (7) are combined to obtain Equation (8) as follows:

$$\frac{{d}^{2} {C}_{T} ( t )}{{dt}^{2}} = ( {K}_{1} {k}_{3} ) {C}_{p} ( t ) + ( {k}_{3} CBV + {k}_{2} CBV + {K}_{1} ) \frac{{dC}_{p} ( t )}{dt} + ( - {k}_{3} - {k}_{2} ) \frac{{dC}_{T} ( t )}{dt} + CBV \frac{{d}^{2} {C}_{p} ( t )}{{dt}^{2}} \qquad ( 8 )$$

Equation (8) is integrated twice to obtain the current activity expression:

$${C}_{T} ( t ) = ( {K}_{1} {k}_{3} ) {\int }_{0}^{t} {\int }_{0}^{τ} {C}_{p} ( s ) dsd τ + ( {k}_{3} CBV + {k}_{2} CBV + {K}_{1} ) {\int }_{0}^{t} {C}_{P} ( τ ) d τ + ( - {k}_{3} - {k}_{2} ) {\int }_{0}^{t} {C}_{T} ( τ ) d τ + CBV \cdot {C}_{p} ( t ) \qquad ( 9 )$$

S2502, a coefficient of each variable in the current activity expression is replaced with each first setting parameter in a first setting parameter set, respectively, to update the current activity expression; wherein the number of first setting parameters in the first setting parameter set is the same as the number of the variables.

Specifically, a first set parameter set is introduced, each parameter of which is expressed as follows:

$${P}_{1} = {k}_{2} + {k}_{3} {P}_{2} = {K}_{1} {k}_{3} {P}_{3} = ( {k}_{2} + {k}_{3} ) CBV + {K}_{1} {P}_{4} = CBV \qquad ( 10 )$$

the coefficient of each variable in the current activity expression is replaced with each first setting parameter in the first setting parameter set (10), Equation (9) is rewritten as:

$${C}_{T} ( t ) = {P}_{1} {\int }_{0}^{t} {C}_{T} ( τ ) d τ + {P}_{2} {\int }_{0}^{t} {\int }_{0}^{τ} {C}_{p} ( s ) dsd τ + {P}_{3} {\int }_{0}^{t} {C}_{P} ( τ ) d τ + {P}_{4} {C}_{p} ( t ) \qquad ( 11 )$$

S2503, flow velocity between tissue compartments corresponding to the PET image set is determined according to the current activity expression.

First, based on the expressions of P₁, P₃, and P₄ in the first set parameter set (10), and the image data corresponding to the PET image set, P₃=P₁P₄+K₁, is obtained, hence K₁=P₃−P₁P₄; this results in a least squares problem min∥EP−C∥, which can be solved using the Lawson-Hanson NNLS (non-negative least squares) algorithm. After quickly obtaining K₁ and CBV through linear computations, CBV is eliminated from Equation (2), and Equation (11) is rewritten accordingly to obtain the following equation:

$${\int }_{0}^{t} {C}_{p} ( τ ) d τ = - {P}_{1} {\int }_{0}^{t} {\int }_{0}^{τ} {C}_{p} ( s ) dsd τ + {P}_{2} {\int }_{0}^{t} {C}_{T}^{*} ( τ ) d τ + {C}_{T}^{*} ( t ) \qquad ( 12 )$$

wherein, C_{T}*(t) is C_{T}*(t)=C_{T}(t)−CBV·Cₚ(t), and K₁, k₂ and k₃ can be calculated from the PET image data and Equation (12).

S2504, net inflow rate of the tracer is determined according to the flow velocity between the tissue compartments and the correlation between the net inflow rate of the tracer and the flow velocity between the tissue compartments.

Wherein, the net inflow rate of the tracer (Kᵢ) is indirectly referred to as the level of the net metabolic rate of glucose in ml/g/min, the correlation between the net inflow rate data of the tracer and the flow velocity between the tissue compartments is

$${K}_{i} = \frac{{K}_{1} {k}_{3}}{{k}_{2} + {k}_{3}} ,$$

and the net inflow rate of the tracer Kᵢ is calculated after obtaining K₁, k₂ and k₃.

The user can select each pixel point of the region of interest to calculate Kᵢ as desired, thus composing a Kᵢ parameter image.

The technical solution of this embodiment determines the net inflow rate of the tracer and the flow velocity between the tissue compartments using an irreversible two-tissue compartmental model, and achieves the determination of PET parameters of the PET images based on the tissue compartmental model and the corresponding activity addition expression using a linear estimation method, further improving the speed of PET parameter determination.

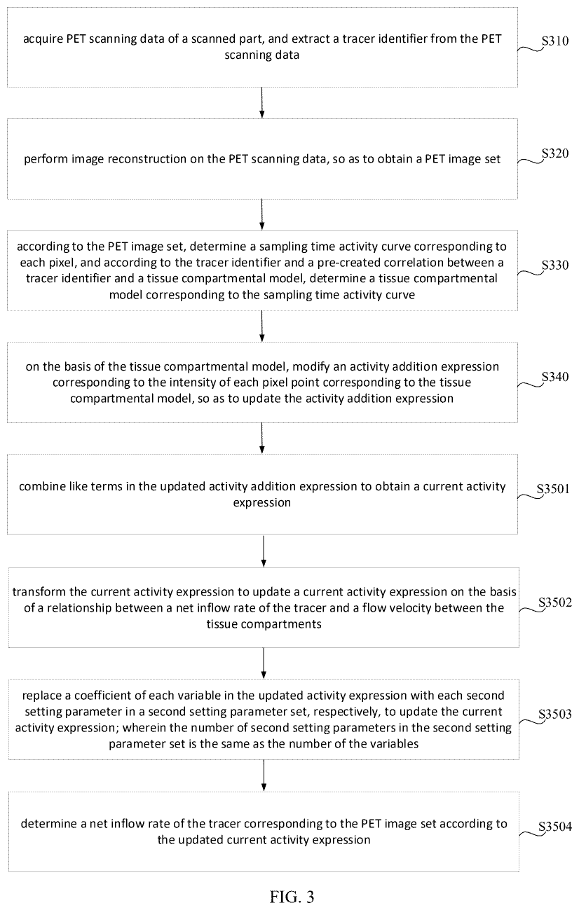

FIG. 3 is a flowchart of yet another PET parameter determination method provided by an embodiment of the present disclosure, which belongs to the same inventive concept as the PET parameter determination method of the previous embodiment, on the basis of which a process of determining a value of at least one dynamic parameter corresponding to a PET image set based on an updated activity addition expression is further performed.

As shown in FIG. 3, the PET parameter determination method includes the following steps:
S310, PET scanning data of a scanned part is acquired, and a tracer identifier is extracted from the PET scanning data.
S320, image reconstruction is performed on the PET scanning data, so as to obtain a PET image set.
S330, according to the PET image set, a sampling time activity curve corresponding to each pixel is determined, and according to the tracer identifier and a pre-created correlation between a tracer identifier and a tissue compartmental model, a tissue compartmental model corresponding to the sampling time activity curve is determined.
S340, on the basis of the tissue compartmental model, an activity addition expression corresponding to the intensity of each pixel point corresponding to the tissue compartmental model is modified, so as to update the activity addition expression.
S3501, like terms in the updated activity addition expression are combined to obtain a current activity expression.

Like terms of Equation (9) are combined to obtain the current activity expression:

$${\int }_{0}^{t} {C}_{T} ( τ ) d τ = \frac{{K}_{1} {k}_{3}}{{k}_{3} + {k}_{2}} {\int }_{0}^{t} {\int }_{0}^{τ} {C}_{p} ( s ) dsd τ + [ \frac{{k}_{3} CBV + {k}_{2} CBV + {K}_{1}}{{k}_{3} + {k}_{2}} ] {\int }_{0}^{t} {C}_{P} ( τ ) d τ + \frac{CBV}{{k}_{3} + {k}_{2}} {C}_{p} ( t ) - \frac{1}{{k}_{3} + {k}_{2}} {C}_{T} ( t ) \qquad ( 13 )$$

S3502, the current activity expression is transformed to update a current activity expression on the basis of a relationship between a net inflow rate of the tracer and a flow velocity between the tissue compartments.
S3503, a coefficient of each variable in the updated activity expression is replaced with each second setting parameter in a second setting parameter set, respectively, to update the current activity expression; wherein the number of second setting parameters in the second setting parameter set is the same as the number of the variables.

Specifically, a second set parameter set is introduced, each parameter of which is expressed as follows:

$${Q}_{1} = \frac{{K}_{1} {k}_{3}}{{k}_{3} + {k}_{2}} {Q}_{2} = \frac{{K}_{1}}{{k}_{3} + {k}_{2}} + CBV {Q}_{3} = \frac{CBV}{{k}_{3} + {k}_{2}} {Q}_{4} = - \frac{1}{{k}_{3} + {k}_{2}} \qquad ( 14 )$$

each second set parameter from the second predetermined parameter set is adopted to replace the coefficients of the respective variables in the current activity expression, and Equation (13) is rewritten as:

$${\int }_{0}^{t} {C}_{T} ( τ ) d τ = {Q}_{1} {\int }_{0}^{t} {\int }_{0}^{τ} {C}_{p} ( s ) dsd τ + {Q}_{2} {\int }_{0}^{t} {C}_{P} ( τ ) d τ + {Q}_{3} {C}_{p} ( t ) + {Q}_{4} {C}_{T} ( t ) \qquad ( 15 )$$

S3504, a net inflow rate of the tracer corresponding to the PET image set is determined according to the updated current activity expression.

First, based on Equation (15) and the image data corresponding to the PET image set, it can be derived that

$${K}_{i} = \frac{{K}_{1} {k}_{3}}{{k}_{2} + {k}_{3}} = {Q}_{1} ,$$

the flow velocity between the tissue compartments for each pixel point is traversed to determine the net inflow rate data Kᵢ of the tracer.

The technical solution of the present embodiment is as follows, the net inflow rate of the tracer is determined using an irreversible two-tissue compartmental model, and on the basis of a tissue compartmental model and a corresponding activity addition expression, a PET parameter of a PET image is determined by using a linear estimation method, thereby improving the speed of estimation of the PET parameter.

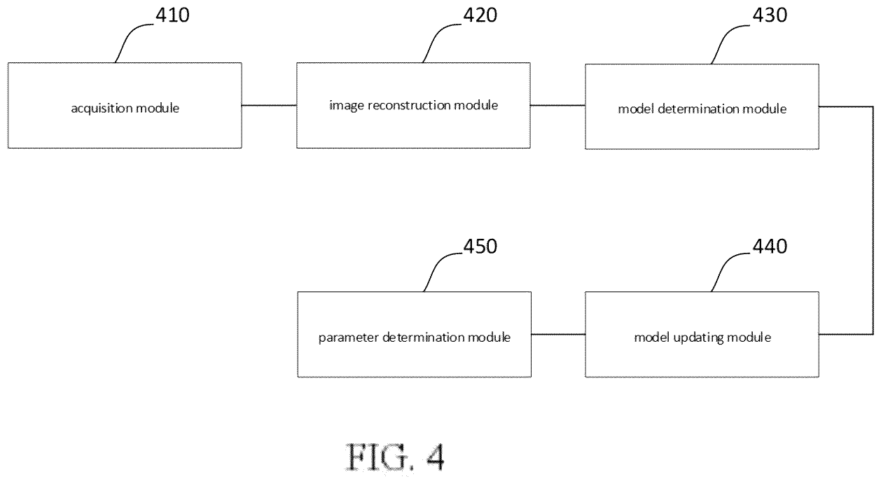

FIG. 4 is a block diagram of a PET parameter determination apparatus according to an embodiment of the present disclosure, which is applicable to a PET parameter determination scenario. The apparatus may be implemented in the form of hardware and/or software, and can be integrated in a computer device having the application development function.

As shown in FIG. 4, the PET parameter determination apparatus includes:
an acquisition module 410, configured to acquire PET scanning data of a scanned part, and extract a tracer identifier from the PET scanning data;
an image reconstruction module 420, configured to perform image reconstruction on the PET scanning data, so as to obtain a PET image set;
a model determination module 430, configured to, according to the PET image set, determine a sampling time activity curve corresponding to each pixel, and according to the tracer identifier and a pre-created correlation between a tracer identifier and a tissue compartmental model, determine a tissue compartmental model corresponding to the sampling time activity curve;
a model updating module 440, configured to, on the basis of the tissue compartmental model, modify an activity addition expression corresponding to the intensity of each pixel point corresponding to the tissue compartmental model, so as to update the activity addition expression; and
a parameter determination module 450, configured to, according to the updated activity addition expression, determine the numerical value of at least one dynamic parameter corresponding to the PET image set, wherein the dynamic parameter includes a flow velocity between tissue compartments in the tissue compartmental model and/or a net inflow rate of a tracer.

Optionally, the parameter determination module 450 is further configured to:
like terms in the updated activity addition expression are combined to obtain a current activity expression;
a coefficient of each variable in the current activity expression is replaced with each first setting parameter in a first setting parameter set, respectively, to update the current activity expression; wherein the number of first setting parameters in the first setting parameter set is the same as the number of the variables;
flow velocity between tissue compartments corresponding to the PET image set is determined according to the current activity expression; and
net inflow rate of the tracer is determined according to the flow velocity between the tissue compartments and the correlation between the net inflow rate of the tracer and the flow velocity between the tissue compartments.

Optionally, the parameter determination module 450 is further configured to:
like terms in the updated activity addition expression are combined to obtain a current activity expression;
the current activity expression is transformed to update a current activity expression on the basis of a relationship between a net inflow rate of the tracer and a flow velocity between the tissue compartments;
a coefficient of each variable in the updated activity expression is replaced with each second setting parameter in a second setting parameter set, respectively, to update the current activity expression; wherein the number of second setting parameters in the second setting parameter set is the same as the number of the variables; and
a net inflow rate of the tracer corresponding to the PET image set is determined according to the updated current activity expression.

Optionally, the apparatus is further configured to determine an image corresponding to the numerical value of the at least one dynamic parameter, respectively, to obtain at least one dynamic parameter image corresponding to the PET image set.

The technical solution of this embodiment is as follows: through the interaction of respective modules, acquiring PET scanning data of a scanned part, and extracting a tracer identifier from the PET scanning data; performing image reconstruction on the PET scanning data, so as to obtain a PET image set; according to the PET image set, determining a sampling time activity curve corresponding to each pixel, and according to the tracer identifier and a pre-created correlation between a tracer identifier and a tissue compartmental model, determining a tissue compartmental model corresponding to the sampling time activity curve; on the basis of the tissue compartmental model, modifying an activity addition expression corresponding to the intensity of each pixel point corresponding to the tissue compartmental model, so as to update the activity addition expression; according to the updated activity addition expression, determining the numerical value of at least one dynamic parameter corresponding to the PET image set, wherein the dynamic parameter includes a flow velocity between tissue compartments in the tissue compartmental model and/or a net inflow rate of a tracer. On the basis of a tissue compartmental model and a corresponding activity addition expression, a PET parameter of a PET image is determined by using a linear estimation method, thereby improving the speed of estimation of the PET parameter.

The PET parameter determination apparatus provided by the embodiments of the present disclosure may perform the PET parameter determination method provided by any one of the embodiments of the present disclosure, with corresponding functional modules and advantageous effects for performing the method.

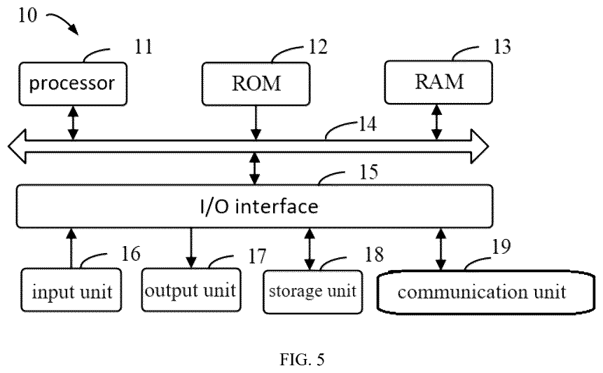

FIG. 5 is a block diagram of an electronic device according to an embodiment of the present disclosure. The electronic device 10 is intended to represent various forms of digital computers, such as laptops, desktops, workstations, personal digital assistants, servers, blade servers, mainframes, or other appropriate computers. The electronic device may also represent various forms of mobile devices, such as personal digital processing, cellular telephones, smart phones, wearable devices (e.g., helmets, glasses, watches, etc.), or other similar computing devices. The components shown herein, their connections, and their functions are meant to be examples only, and are not meant to limit implementations of the disclosures described and/or claimed in this document.

As shown in FIG. 5, the electronic device 10 includes at least one processor 11, and a memory communicatively connected with the at least one processor 11, such as a read only memory (ROM) 12, a random access memory (RAM) 13, etc., in which the memory stores a computer program executable by at least one processor, the processor 11 may perform various appropriate actions and processes in accordance with the computer program stored in the read only memory (ROM) 12 or loaded into the random access memory (RAM) 13 from the storage unit 18. In the RAM 13, various programs and data required for the operation of the electronic device 10 can also be stored. The processor 11, the ROM 12 and the RAM 13 are connected to each other by a bus 14. An input/output (I/O) interface 15 is also connected to the bus 14.

A number of components in the electronic device 10 are connected to the I/O interface 15, including: an input unit 16, such as a keyboard or mouse; an output unit 17, such as various types of displays or speakers or the like; a storage unit 18, such as a magnetic or optical disk, etc.; and a communication unit 19, such as a network card, a modem, or a wireless communication transceiver. The communication unit 19 allows the electronic device 10 to exchange information/data with other devices through computer networks such as the Internet and/or various telecommunication networks.

The processor 11 may be various general-purpose and/or special-purpose processing components having processing and computing capabilities. Some examples of the processor 11 include, but are not limited to, a central processing unit (CPU), a graphics processing unit (GPU), various specialized artificial intelligence (AI) computing chips, various processors running machine learning model algorithms, a digital signal processor (DSP), any suitable processor, controller or microcontroller, or the like. The processor 11 performs the various methods and processes described above, such as the PET parameter determination method.

In some embodiments, the PET parameter determination method may be implemented as a computer program tangibly embodied on a computer readable storage medium, such as storage unit 18. In some embodiments, part or all of the computer program may be loaded into and/or installed onto the electronic device 10 via the ROM 12 and/or the communication unit 19. When the computer program is loaded into the RAM 13 and executed by the processor 11, one or more steps of the PET parameter determination method described above may be performed. Alternatively, in other embodiments, the processor 11 may be configured to perform the PET parameter determination method in any other suitable manner, e.g., by means of firmware.

Various implementations of the systems and techniques described above here can be realized in digital electronic circuit systems, integrated circuit systems, Field-Programmable Gate Arrays (FPGAs), Application-Specific Integrated Circuits (ASICs), Application-Specific Standard Products (ASSPs), Systems on Chip (SoCs), Complex Programmable Logic Devices (CPLDs), computer hardware, firmware, software, and/or combinations thereof. These various embodiments may include: being implemented in one or more computer programs that can be executed and/or interpreted on a programmable system including at least one programmable processor, which can be a dedicated or general-purpose programmable processor. This processor is capable of receiving data and instructions from a storage system, at least one input device, and at least one output device, and transmitting data and instructions to the storage system, the at least one input device, and the at least one output device.

Computer programs for carrying out methods of the present disclosure may be written in any combination of one or more programming languages. These computer programs may be provided to a processor of a general purpose computer, a special purpose computer, or other programmable data processing apparatuses, such that the computer programs, when executed by the processor, cause the functions/operations specified in the flowcharts and/or block diagrams to be implemented. The computer program may execute entirely on a machine or partially on a machine, partially on a machine as a stand-alone software package and partially on a remote machine or entirely on a remote machine or server.

In the context of the present disclosure, a computer readable storage medium may be a tangible medium that can contain, or store a computer program for use by or in connection with an instruction execution system, apparatus, or device. A computer readable storage medium may include, but is not limited to, an electronic, magnetic, optical, electromagnetic, infrared, or semiconductor system, apparatus, or device, or any suitable combination of the foregoing. Alternatively, a computer readable storage medium may be a machine readable signal medium. A more specific example of the machine readable storage medium would include an electrical connection based on one or more wires, a portable computer diskette, a hard disk, a random access memory (RAM), a read-only memory (ROM), an erasable programmable read-only memory (EPROM or Flash memory), an optical fiber, a convenient compact disc read-only memory (CD-ROM), an optical storage device, a magnetic storage device, or any suitable combination of the foregoing.

To provide for interaction with a user, the systems and techniques described here can be implemented on an electronic device having a display device (e.g., a CRT (cathode ray tube) or LCD (liquid crystal display) monitor) for displaying information to the user; and a keyboard and a pointing device (e.g., a mouse or trackball) by which a user can provide input to an electronic device. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes back-end components (e.g., as a data server), or a computing system that includes a middleware component (e.g., an application server), or a computing system that includes a front-end component (e.g., a user computer having a graphical user interface or a Web browser through which a user can interact with an implementation of the systems and techniques described here), or a computing system that includes any combination of such back-end component, middleware, or front-end component. The components of the system can be interconnected by any form or medium of digital data communication (e.g., a communication network). Examples of the communication network include a local area network (LAN), a wide area network (WAN), a block chain network, and the Internet.

The computing system can include clients and servers. The client and server are generally remote from each other and typically interact through a communication network. The relationship of the client and server arises by virtue of computer programs running on the respective computers and having a client-server relationship to each other. The server may be a cloud server, also referred to as a cloud computing server or a cloud host, and is a host product in a cloud computing service architecture to solve the drawbacks of the conventional physical host and VPS services, which are difficult to manage and weak to scale.

It should be understood that steps may be reordered, added, or deleted using the various flow forms shown above. For example, the steps described in the present disclosure may be executed in parallel, sequentially, or in different orders, and are not limited herein as long as the desired results of the aspect of the present disclosure can be achieved.

The foregoing detailed implementations should not be construed as limiting the scope of the present disclosure. It should be understood by those skilled in the art that various modifications, combinations, sub-combinations and substitutions can occur depending on design requirements and other factors. It is intended that all such modifications, equivalents, modifications, and the like, which fall within the spirit and principles of the present disclosure, be included within the scope of the present disclosure.

What is claimed is:

1. A PET parameter determination method, comprising:
acquiring PET scanning data of a scanned part, and extracting a tracer identifier from the PET scanning data;
performing image reconstruction on the PET scanning data, so as to obtain a PET image set;
according to the PET image set, determining a sampling time activity curve corresponding to each pixel, and according to the tracer identifier and a pre-created correlation between a tracer identifier and a tissue compartmental model, determining a tissue compartmental model corresponding to the sampling time activity curve;
on the basis of the tissue compartmental model, modifying an activity addition expression corresponding to the intensity of each pixel point corresponding to the tissue compartmental model, so as to update the activity addition expression; and
according to the updated activity addition expression, determining the numerical value of at least one dynamic parameter corresponding to the PET image set, wherein the dynamic parameter comprises a flow velocity between tissue compartments in the tissue compartmental model and/or a net inflow rate of a tracer.

2. The method according to claim 1, wherein according to the updated activity addition expression, determining the numerical value of at least one dynamic parameter corresponding to the PET image set comprises:
combining like terms in the updated activity addition expression to obtain a current activity expression;
replacing a coefficient of each variable in the current activity expression with each first setting parameter in a first setting parameter set, respectively, to update the current activity expression;
wherein the number of first setting parameters in the first setting parameter set is the same as the number of the variables;
determining flow velocity between tissue compartments corresponding to the PET image set according to the current activity expression; and
determining net inflow rate of the tracer according to the flow velocity between the tissue compartments and the correlation between the net inflow rate of the tracer and the flow velocity between the tissue compartments.

3. The method according to claim 1, wherein according to the updated activity addition expression, determining the numerical value of at least one dynamic parameter corresponding to the PET image set comprises:
combining like terms in the updated activity addition expression to obtain a current activity expression;
transforming the current activity expression to update a current activity expression on the basis of a relationship between a net inflow rate of the tracer and a flow velocity between the tissue compartments;
replacing a coefficient of each variable in the updated activity expression with each second setting parameter in a second setting parameter set, respectively, to update the current activity expression; wherein the number of second setting parameters in the second setting parameter set is the same as the number of the variables; and
determining a net inflow rate of the tracer corresponding to the PET image set according to the updated current activity expression.

4. The method according to claim 1, wherein the tracer identifier is 18FDG and the tissue compartmental model is an irreversible two-tissue compartmental model.

5. The method according to claim 1, wherein,
the tissue compartmental model is a reversible one-tissue compartmental model.

6. The method according to claim 1, wherein
the image in the PET image set is an image with a standard uptake value.

7. The method according to claim 1, further comprising:
determining an image corresponding to the numerical value of the at least one dynamic parameter, respectively, so as to obtain at least one dynamic parameter image corresponding to the PET image set.

8. A PET parameter determination apparatus, comprising:
an acquisition module, configured to acquire PET scanning data of a scanned part, and extract a tracer identifier from the PET scanning data;
an image reconstruction module, configured to perform image reconstruction on the PET scanning data, so as to obtain a PET image set;
a model determination module, configured to, according to the PET image set, determine a sampling time activity curve corresponding to each pixel, and according to the tracer identifier and a pre-created correlation between a tracer identifier and a tissue compartmental model, determine a tissue compartmental model corresponding to the sampling time activity curve;
a model updating module, configured to, on the basis of the tissue compartmental model, modify an activity addition expression corresponding to the intensity of each pixel point corresponding to the tissue compartmental model, so as to update the activity addition expression; and
a parameter determination module, configured to, according to the updated activity addition expression, determine the numerical value of at least one dynamic parameter corresponding to the PET image set, wherein the dynamic parameter comprises a flow velocity between tissue compartments in the tissue compartmental model and/or a net inflow rate of a tracer.

9. An electronic device, comprising:
at least one processor; and
a memory communicatively connected with the at least one processor; wherein,
the memory stores a computer program executable by the at least one processor, the computer program being executed by the at least one processor to enable the at least one processor to perform the PET parameter determination method according to claim 1.

10. A computer-readable storage medium having stored thereon computer instructions for causing a processor to implement the PET parameter determination method according to claim 1 when executed.
